(19) United States
(12) Patent Application Publication
(10) Pub. No.: US 2020/0069072 A1
(43) Pub. Date: Mar. 5, 2020

(54) Diaper Changing System and Method

(71) Applicants: Edward Agcaoili, Katy, TX (US); Bernadine Agcaoili, Katy, TX (US)

(72) Inventors: Edward Agcaoili, Katy, TX (US); Bernadine Agcaoili, Katy, TX (US)

(21) Appl. No.: 16/522,464

(22) Filed: Jul. 25, 2019

Related U.S. Application Data
(60) Provisional application No. 62/725,658, filed on Aug. 31, 2018.

(51) Int. Cl. A47D 5/00; A61F 13/551

(52) U.S. Cl. CPC A47D 5/006; A61F 13/55115; A61F 2013/8402; A61F 13/5512; A61F 13/5519

(57) ABSTRACT

The application is directed to a system for changing out a used undergarment worn by a person. The system includes an outer package comprising a changing pad and one or more changing items therein. A person may be placed atop the changing pad during changing out from the used undergarment to a new or clean undergarment. The changing pad may be used as a disposal package for the outer package, used undergarment and the one or more changing items.

### CROSS-REFERENCE TO RELATED APPLICATIONS

This application claims benefit of U.S. Provisional Patent Application Ser. No. 62/725,658, filed on Aug. 31, 2018, the content of which is hereby incorporated by reference in its entirety.

### STATEMENT REGARDING FEDERALLY SPONSORED RESEARCH OR DEVELOPMENT

Not applicable.

### BACKGROUND OF THE APPLICATION

### 1. Field of the Invention

The application relates generally to a diaper changing system and method.

### 2. Background Art

The changing of diapers for babies, small children and incontinent adults is often a challenge away from home because caregivers often lack one or more basic changing items effective for changing a diaper in a hygienic or sanitary manner. Attempts have been made to provide portable diaper changing kits. However, known portable diaper changing kits provide changing items in a cumbersome bulky packaging that is inconvenient for transport and/or lack one or more changing items that are commonly used at home when changing diapers. For example, known kits do not provide multi-purpose changing items for hygienic or sanitary diaper changing and disposal of the changing items. Also, known kits are provided with standard sized diapers for a target child or incontinent adult. If a caretaker is changing diapers for two or more persons, various size diapers may have to be carried outside the home.

Overcoming the above shortcomings is desired.

### SUMMARY

The application is directed to a system for changing out a soiled absorbent article worn by a person including outer packaging and a vacuum shrunk clean absorbent article housed within the outer packaging.

The application is also directed to a diaper changing system including (a) an outer packaging; (b) one or more diaper changing accessory items housed therein including at least a diaper changing pad and a vacuum shrunk adjustable diaper, wherein the diaper changing pad is operationally configured as a post diaper change disposal bag for disposal of at least the outer packaging and a soiled diaper.

The application is also directed to a method of changing a diaper including the following steps: (a) providing a diaper changing system including an outer packaging and items housed therein including at least an adjustable diaper, a diaper changing pad and one or more wiping type items; (b) removing the items from the outer packaging and placing the diaper changing pad on a support surface in an open position; (c) placing a child wearing a used diaper atop the diaper changing pad; (d) removing the used diaper and cleaning the child using the one or more wiping type items; (e) placing the adjustable diaper on the child in a fitted manner; and (f) sealing the outer packaging, the used diaper and one or more wiping type items within the diaper changing pad for disposal.

### BRIEF DESCRIPTION OF THE SEVERAL VIEWS OF THE DRAWINGS

FIG. 1 is a perspective view of a simplified embodiment of a sealed changing system of this application showing an outer packaging of the system.

FIG. 2 is a partial phantom perspective view of an embodiment of a sealed changing system including various changing system accessory items enclosed within an outer packaging.

FIG. 3 is an illustration of various changing system accessory items that may be included as part of a changing system.

FIG. 4 is a side phantom view of a simplified sealed changing system including a diaper changing pad and various changing system accessory items enclosed within an outer packaging.

FIG. 5 is a top view of a foldable diaper changing pad.

FIG. 6 is a perspective view of an embodiment of a sealable disposal bag of the present changing system.

FIG. 7 is a partial phantom perspective view of an embodiment of outer packaging of the present changing system.

FIG. 8 is another partial phantom view of the outer packaging of FIG. 7 including a detachable section set at a partially open position.

FIG. 9 is a top view of an embodiment of a folded diaper changing pad.

FIG. 10 is a top view of the diaper changing pad of FIG. 9 in an open unfolded position.

FIG. 11 is a top view of another embodiment of a diaper changing pad.

FIG. 12 is a top view of the diaper changing pad of FIG. 11 in an open unfolded position.

FIG. 13 is a top partial phantom view of the diaper changing pad of FIG. 11 in a folded position post diaper change.

FIG. 14 is a partial phantom view of the diaper changing pad of FIG. 11 in an inverted position.

FIG. 15 is a simplified illustration of an adjustable diaper system including a main body and a removable member.

FIG. 16 is a depiction of a main body of the adjustable diaper system of FIG. 15 shown in a split position.

FIG. 17 is a simplified illustration of the adjustable diaper system of FIG. 15 depicting a removable member secured to the main body.

FIG. 18 is a depiction of another embodiment of a main body of an adjustable diaper system.

FIG. 19 is a depiction of the adjustable diaper system including the main body of FIG. 18.

FIG. 20 is a simplified illustration of a removable member of the adjustable diaper system of FIG. 19.

FIG. 21 is a top view of another embodiment of a diaper in an unadjusted position.

FIG. 22 is a top view of the adjustable diaper of FIG. 21 shown in an adjusted position.

### DETAILED DESCRIPTION

It has been discovered that a diaper changing system including an adjustable diaper and one or more multi-use changing items may be provided in a relatively convenient small carry package while providing substantially all of the materials desired for changing an individual's diaper, cleaning an individual and post-changing sanitary disposal thereof Heretofore, such a desirable achievement has not been considered possible, and accordingly, the diaper changing system and method of this application measure up to the dignity of patentability and therefore represents a patentable concept.

Before describing the invention in detail, it is to be understood that the present changing system and method are not limited to particular embodiments. It is also to be understood that the terminology used herein is for the purpose of describing particular embodiments only, and is not intended to be limiting. Herein, the phrase “vacuum package” refers to removing substantially all of the air from a particular package prior to sealing the package and its contents therein. The term “caregiver” refers to a person responsible for changing the diaper of a child or incontinent adult. Herein, the term “child” may refer to a baby of up to about twelve months of age and/or a toddler from about one to three years of age. The term “carrier” refers to a device used by a caregiver or other individual for carrying various items. Suitable carriers may include, but are not necessarily limited to bags, sacks, purses, backpacks, satchels, luggage, pouches, rucksacks, cases, binders, notebook type cases, lunch boxes, and other toteable objects and devices. The terms “sanitary” and “hygienic” may be used interchangeably and refer to the containment and disposal of biological waste without contacting the skin or other surfaces. As used herein the term “absorbent article” refers to articles placed against or in proximity to the body of a wearer to absorb and contain various exudates discharged from the body. The term “disposable” refers to articles which are generally intended to be discarded after a single use (i.e., they are not generally intended to be laundered or otherwise restored or reused). As used herein, the term “diaper” refers to a garment generally worn by children and/or incontinent persons about the lower torso to receive and contain bodily exudates. The present application is also applicable to other disposable absorbent articles such as incontinence briefs, undergarment absorbent inserts, disposable training pants, belted diapers, diaper holders, sanitary napkins, and the like.

In one aspect, the application provides a portable sealable diaper changing system including an outer packaging material, a vacuum packaged reduced size adjustable absorbent article and one or more other changing items or accessories packaged therein for commercial use.

In another aspect, the application provides a diaper changing system including one or more child diaper related changing items sealed within a pre-change bag member in a tamper proof manner, the system including at least a vacuum shrinkable adjustable diaper and a post-change disposal bag member. In one embodiment the post-change bag member may also operate as a diaper changing pad.

In another aspect, the application provides a child diaper changing system or kit including a vacuum sealed reduced size diaper for ease of transport in a carrier.

In another aspect, the application provides a diaper changing system for incontinent adults including one or more adult hygienic accessories sealed within a pre-change bag member.

In another aspect, the application provides a diaper changing system including an adjustable absorbent article for incontinent adults.

In another aspect, the application provides a system for providing feminine protection articles in a pre-shrunk packaging.

In another aspect, the application provides a sealed diaper type changing system including a vacuum sealed reduced size diaper therein, wherein the diaper is operationally configured to return to its original size when its separate shrink wrap type packaging is opened, i.e., when the vacuum seal of the packaging is broken.

In another aspect, the application provides a diaper changing system including outer packaging operationally configured to seal original contents including a reduced size adjustable absorbent article and one or more other changing materials therein including a diaper changing pad operationally configured to seal a used diaper and the other changing materials therein for sanitary disposal of the original contents and outer packaging. In another embodiment, it is contemplated that the outer packaging itself may be used as a diaper changing pad wherein the system includes a separate disposal bag as part of the original content of the system.

In another aspect, the application provides a changing system including an outer packaging member and a reduced size adjustable absorbent article and related changing items or accessories housed therein. The packaging member is suitably easily carried by an individual and is easily displayed and/or stacked on a store shelf or the like.

In another aspect, the application provides a system for use in the act of changing diapers, wherein the system includes an outer packaging and various content including at least an adjustable absorbent article and wherein the sealed packaging may be stored and dispensed from a vending machine or the like.

In one aspect, a child changing system is provided including one or more child changing items sealed within a pre-change bag member, the child changing items including at least a shrinkable adjustable diaper and a post-change bag member operationally configured to act as (a) a child changing pad and (b) a disposable bag for sealing changing system items and a soiled diaper therein for disposal purposes.

In another aspect, a child changing system is provided including an outer package member housing a vacuum sealed shrinkable adjustable diaper and one or more child changing items including at least a multi-purpose bag member operationally configured for use as a child changing pad and a disposal bag that is operationally configured to seal the outer package member, a used diaper, and child changing items therein for disposal.

In another aspect, a child changing system is provided including a pre-change bag member constructed from fluid sealable material operationally configured to seal one or more child changing items therein, the one or more child changing items including at least (1) an adjustable diaper that may be compressed from a first size to a second size under vacuum and (2) a foldable changing pad operationally configured to seal child changing system items and a used diaper therein for disposal purposes. The application is also directed to a system including a changing pad having a fluid adsorbing layer including a layer or matrix of fluff material with super adsorbent polymer (“SAP”) as understood by persons of ordinary skill in the art of diapers.

In another aspect, a child diaper changing system is provided including a vacuum sealed package housing a diaper and one or more changing accessory items, wherein the bag is operationally configured to shrink the diaper under vacuum to a size about one-tenth the diaper's operating size. In one embodiment, a shrinkable adjustable diaper includes a first size when vacuum sealed within the first bag member and a second size when the seal of the first bag member is broken.

In another aspect, the application is directed to a post-change sealable bag member having (1) a foldable member including a first section and a second section that unfolds to provide a substantially planar changing pad, (2) one or more absorbent members disposed across portions of the first and section sections, (3) an adhesive strip assembly on the first section and (4) a pull strip member on the first section to assist in sealing the remaining changing system items and a used diaper therein for disposal purposes. The post-change bag member may include a first usable surface area in a folded position and a second usable surface area in an unfolded position. The second usable surface area is up to about twice the surface area as the first usable surface area.

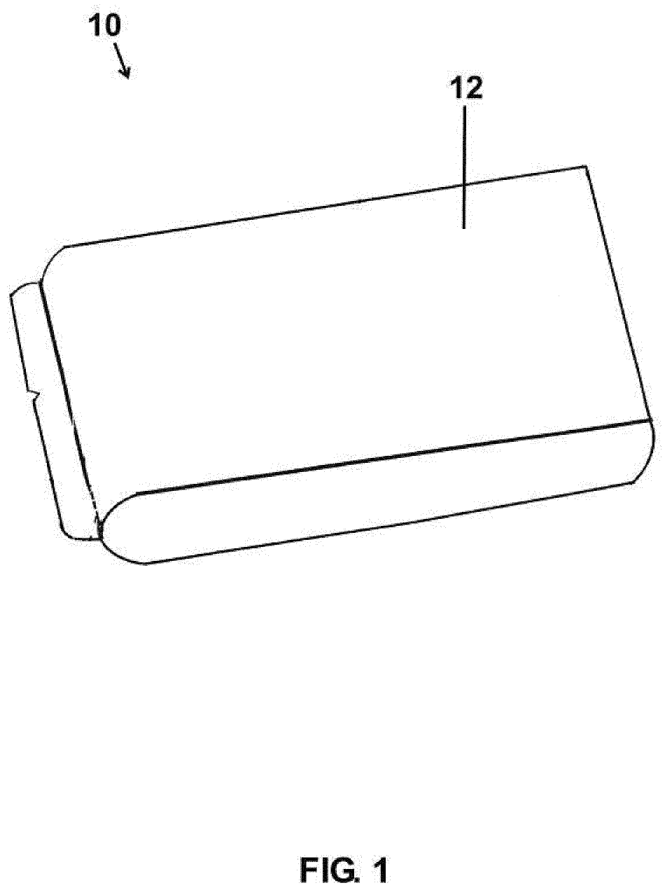

In one simplified embodiment as shown in FIG. 1, the changing system (hereafter “system” 10) of this application may include an outer pre-change package member or “outer packaging 12” operationally configured to seal an absorbent article such as a diaper and/or one or more diaper changing accessory items (hereafter “child changing items”) therein. Depending on the intended use of the system 10, the absorbent article and/or or more child changing items may be sterilized and sealed with an outer packaging 12.

For ease of storage and transport, the outer packaging 12 may also be provided as a vacuum sealed outer member effective for minimizing the size of the system 10. As such, a suitable absorbent article of the system 10, e.g., a diaper, is reducible in size under vacuum to a shrunken or reduced size of about 55.0 percent its resting size providing an outer packaging 12 of a convenient reduced size.

Without limiting the invention, a suitable sealable outer packaging 12 may be constructed from one or more flexible waterproof materials. Suitable outer packaging 12 materials include, but are not necessarily limited to plastics, biodegradable plastics (or “bioplastics”), and combinations thereof. Suitable plastics include, but are not necessarily limited to thermoplastic polymers, e.g., polyethylene. One suitable biodegradable outer packaging 12 may be constructed from polylactic acid (“PLA”) designed to break down in compost systems. Another biodegradable outer packaging 12 may be constructed from a combination of thermoplastic starch and polyolefins. Such materials may be employed alone or in combination with one or more other materials such as aliphatic polyamides, e.g., nylon, effective to seal an absorbent article, one or more changing items for providing a commercially viable system 10.

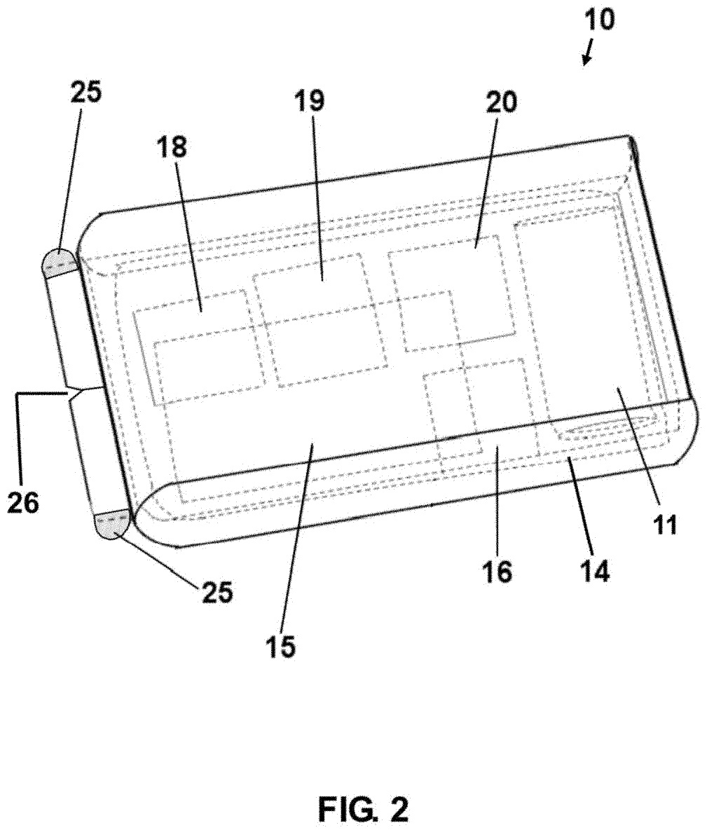

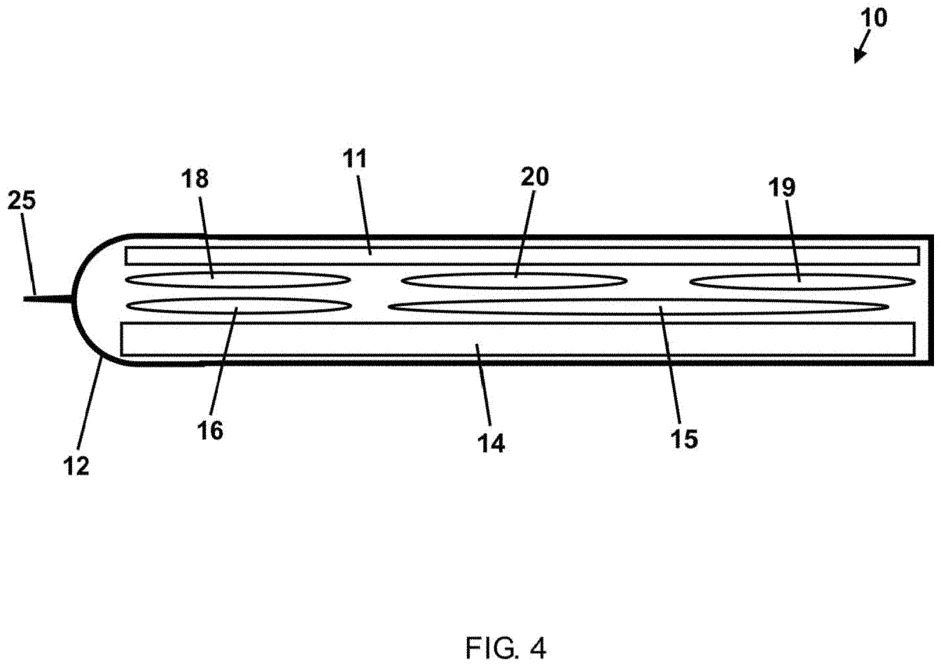

Suitably, the system 10 may include at least one fixed size or adjustable diaper 14, one or more pre-packaged pre-moistened wipes 15, one or more skin care items such as a pre-packaged ointment 16 or the like, a disposal changing pad 11, pre-packaged sanitizer 18, pre-packaged skin lotion 19, a pre-packaged powder 20, and combinations thereof. Suitably, the system 10 may include one or more changing item accessories provided in individual disposable easy tear packaging sealable within the outer packaging 12 in a manner effective to minimize space requirements of the system 10 by reducing the size of the diaper 14 under vacuum. As such, a system 10 as shown in FIGS. 1-4 is suitable for transport in a carrier whereby the vacuum shrunk diaper 14 is operationally configured to return to its normal larger pre-shrunk resting size once the vacuum seal of its separate packaging is broken.

Still referring to FIGS. 1-4, one suitable embodiment of the system 10 may include a plastic outer packaging 12 that may be sealed and house a vacuum sealed clean absorbent article (e.g., an adjustable diaper 14) and one or more child changing items 13 therein. Although the needs and/or wants of a particular caregiver and/or a particular diaper wearer may vary, exemplary child changing items for the purposes of this application may include, but are not necessarily limited to a diaper changing pad, one or more cleaning or wiping type items, one or more drying type items, one or more powder(s), one or more skin care items, one or more disposable bag type items, caregiver gloves, and combinations thereof. Suitable cleaning or wiping type items may include, but are not necessarily limited to liquid sanitizers, wet cloths, wet pads, pre-moistened wipes, and combinations thereof. Suitable drying type items may include, but are not necessarily limited to towels, dry wipes, dry clothes, towelletes, napkins, dry pads, tissue paper, sponges, and combinations thereof. Suitable skin care items may include, but are not necessarily limited to skin lotions, ointments, baby ointments, talcum powder, baby powder, disinfectants, medicinal wet wipes, and combinations thereof. Suitable medicinal wet wipes may include one or more medicinal constituents including, but not necessarily limited to baby rash ointment(s), hemorrhoid ointment(s), and combinations thereof. Suitable gloves may include, but are not necessarily limited to one or more latex gloves, nitrile gloves, or the like. As understood by the skilled artisan, the system 10 may include a pair of gloves. Other accessories may also be provided as desired, e.g., cotton swabs, perfume, nursing items, baby food or baby formula in powder form to be reconstituted by addition of water, and combinations thereof

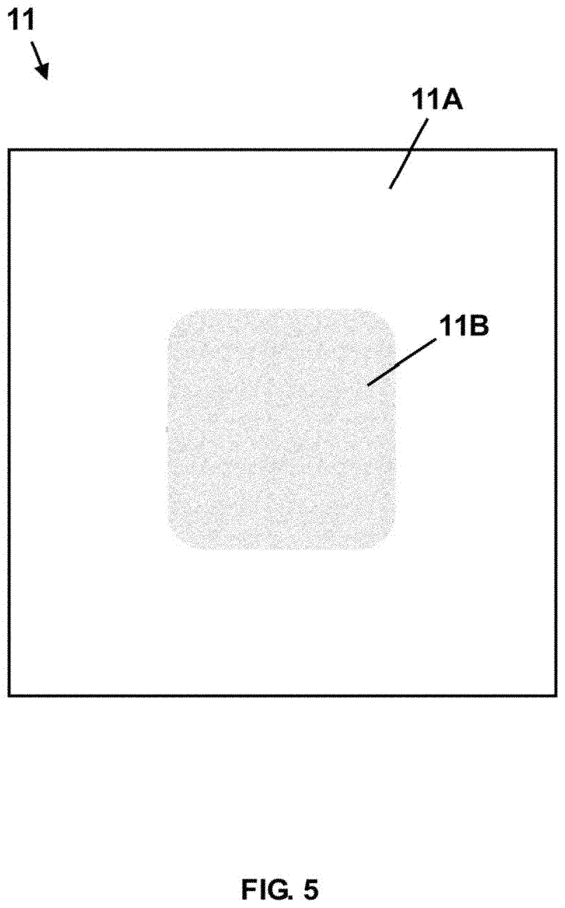

In one embodiment of a system 10 for child use, the system 10 may include a foldable changing pad 11 operationally configured as a changing surface and a stand-alone disposal bag for hygienic disposal of the outer packaging 12, the one or more child changing items and a soiled diaper. One suitable changing pad 11 may include a foldable planar type member with at least a first fluid impermeable layer 11A and a second fluid adsorbing layer 11B attached to the first fluid impermeable layer 11A. As shown in the simplified illustration of FIG. 5, the second fluid adsorbing layer 11B may be centrally located along an upper surface of the first fluid impermeable layer 11A although other locations may be utilized for one or more particular operations. In another embodiment, two or more second fluid adsorbing layer 11B may be disposed along a first fluid impermeable layer 11A as desired.

As shown in FIG. 5, a suitable second fluid adsorbing layer 11B includes one or more adsorbent materials of a surface area (1) effective to adsorb any fluids realized during the changing of a child's diaper 14 and/or (2) effective to support the buttocks of a child thereon. Although the second fluid adsorbing layer 11B may cover the entire first fluid impermeable layer 11A, to minimize manufacturing costs a suitable foldable changing pad 11 is provided with a second fluid adsorbing layer 11B covering from about five percent to about seventy-five percent the surface of the first fluid impermeable layer 11A. Without limiting the scope of the invention, a suitable foldable changing pad 11 to be provided within the outer packaging 12 includes the following dimensions:
(a)—Length of the first fluid impermeable layer 11A:
   about 30.48 cm to about 76.2 cm (about 12.0 inches to about 30.0 inches)
(b)—Width of the first fluid impermeable layer 11A:
   about 25.4 cm to about 76.2 cm (about 10.0 inches to about 30.0 inches)
(c)—Thickness of the first fluid impermeable layer 11A:
   about 0.1 mm to about 1.0 mm (about 0.004 inches to about 0.04 inches);
(d)—Thickness of the second fluid adsorbing layer 11B:
   about 1.0 mm to about 5.0 mm (about 0.04 inches to about 0.2 inches).

Even though the first fluid impermeable layer 11A is shown in FIG. 5 as having a rectangular shape defined by corners having right angles, in another embodiment the first fluid impermeable layer 11A may have rounded corners or include a circular shape, an oval shape, a triangular shape or other multi-sided shape, or an irregular shape. Likewise, although the second fluid adsorbing layer 11B is shown as having a rectangular shape with rounded corners, in another embodiment the second fluid adsorbing layer 11B may include right angle corners or include a circular shape, an oval shape, a triangular shape or other multi-sided shape, or an irregular shape.

In another embodiment of the system 10 for child use, the system 10 may include a foldable changing pad 11 and a separate stand-alone sealable disposable bag for hygienic disposal of the outer packaging 12, the one or more child changing items and a soiled diaper. One suitable sealable disposal bag is operationally configured to maintain disposal contents within the sealed disposal bag and/or prevent odor(s) from emitting out from the disposal bag once sealed. Without limiting the scope of the invention, one suitable sealable disposal bag 21 to be included as part of the system 10 may include a sealable envelope type configuration as shown in FIG. 6.

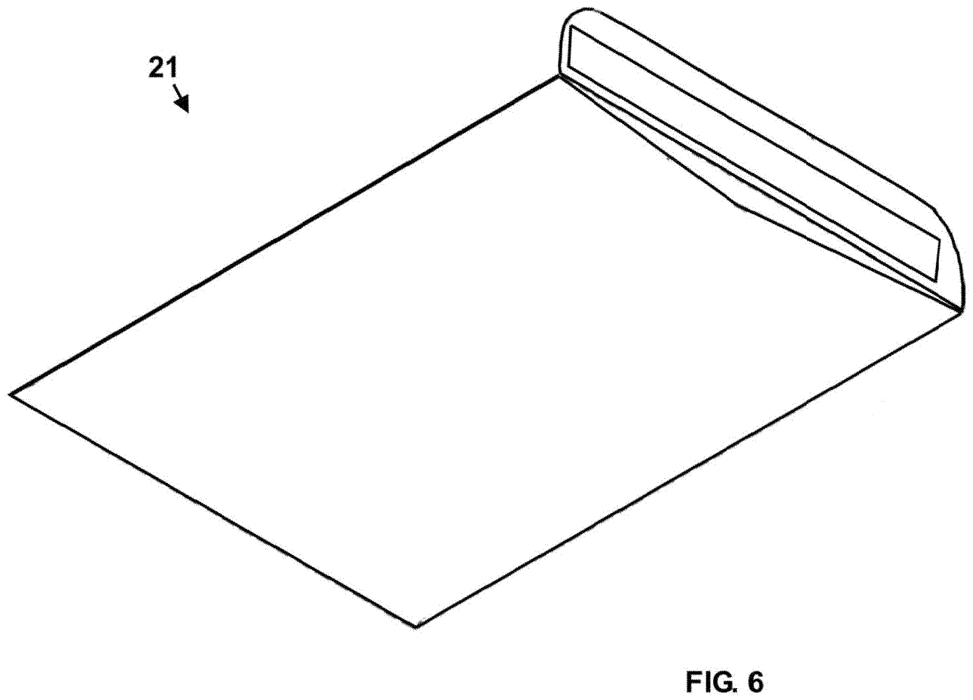

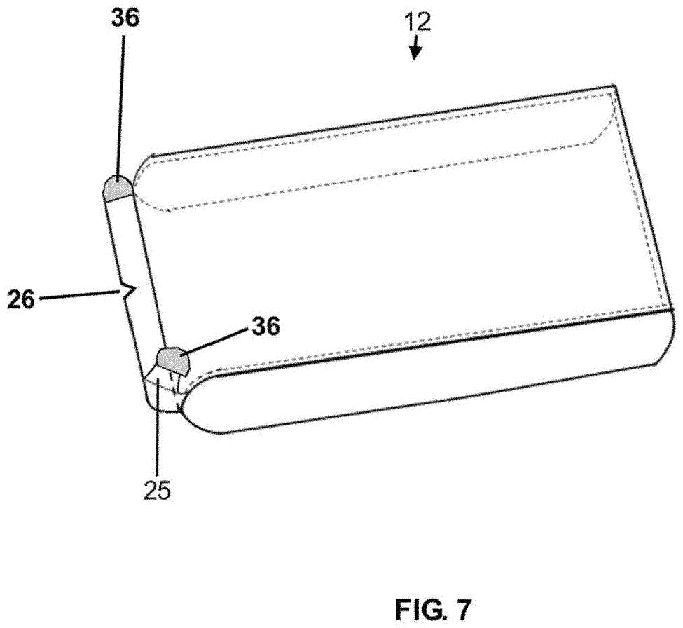

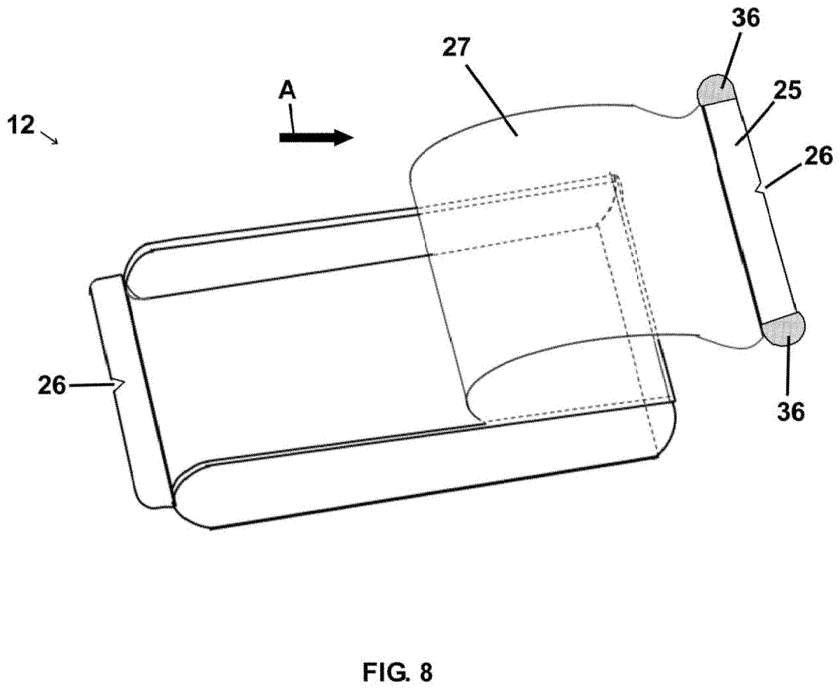

As is typical to product packaging, an outer packaging 12 may be opened as desired, e.g., torn open with hands and/or teeth, cut open. As shown in FIG. 7, one suitable outer packaging 12 of this type may include a peel-back member 25 with one or more pull tab members 36 and/or a tear-off notch 26 for ease of opening the outer packaging 12. Although a tear-off notch 26 may be located along the outer packaging 12 as desired, in one suitable embodiment the tear-off notch 26 may be incorporated into the peel-back member 25 as shown—suitably near an edge of the outer packaging 12. With attention to FIG. 8, the peel-back member 25 may include a two-part wing assembly whereby a first part of the peel-back member 25 is incorporated into the outer packaging 12 and an opposite second part of the peel-back member 25 is incorporated into a detachable section 27 that may be peeled off from the remaining outer packaging 12 according to directional arrow A as shown. In operation, the first part of the peel-back member 25 may be held with one hand while the second part may be pulled apart from the first part exposing the contents of the system 10. As shown, the outer packaging 12 may be operationally configured so that the detachable section 27 releases from the outer packaging 12 along its side edges according to a perforated edge or the like. Other peel-back configurations are also contemplated as understood by those of ordinary skill in the art of product packaging. In another embodiment, outer packaging 12 may include a perforated type lateral tear edge as is common in commercial packaging of disposable household items. Although the system 10 may be built to scale, one suitable outer packaging 12 for commercial use may include the following dimensions:
Length: about 20.32 cm (8.0 inches);
Width: about 12.7 cm (5.0 inches); and
Thickness: about 2.54 cm (1.0 inch).

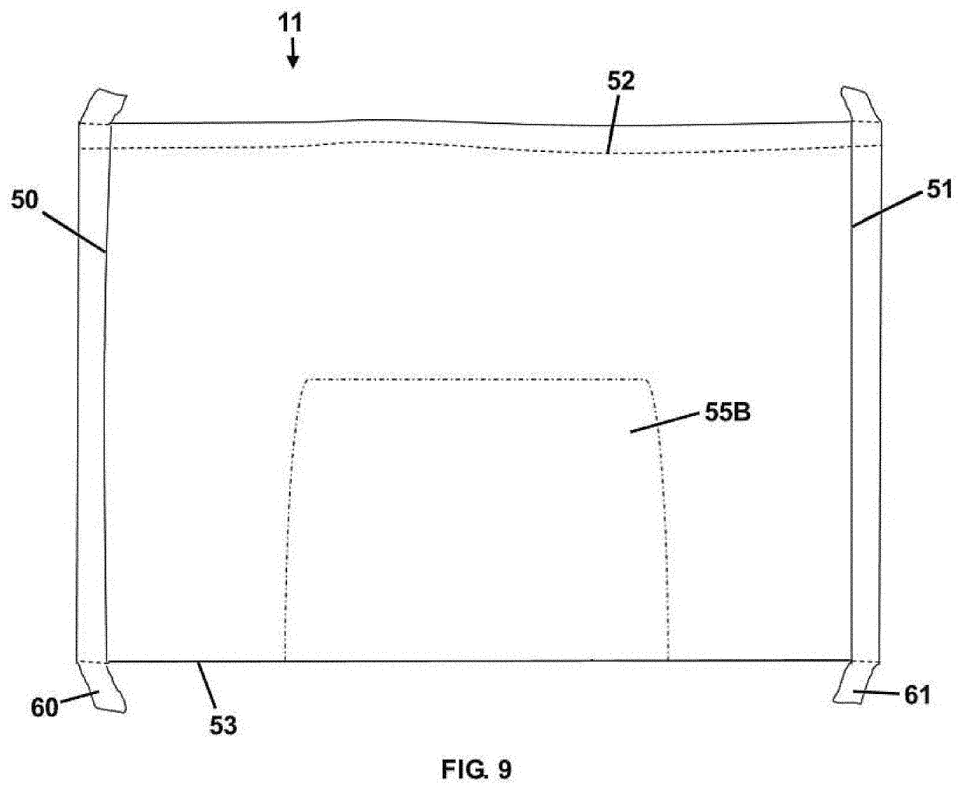

In still another embodiment the system 10 may include a foldable changing pad 11 that further functions as a post diaper change re-sealable bag for hygienic disposal of the outer packaging 12 as well as the changing items and a soiled diaper sealed within the outer packaging 12. In this embodiment, once the changing pad 11 is sealed, the changing pad 11 is operationally configured to maintain disposal contents therein and/or prevent odor(s) from emitting out therefrom. As shown in FIG. 9, one suitable changing pad 11 may include a planar rectangular type member foldable in half (or including additional folds) and operationally configured to be sealed about its three accessible sides 50, 51, and 52 at or near the edges of each of the three sides.

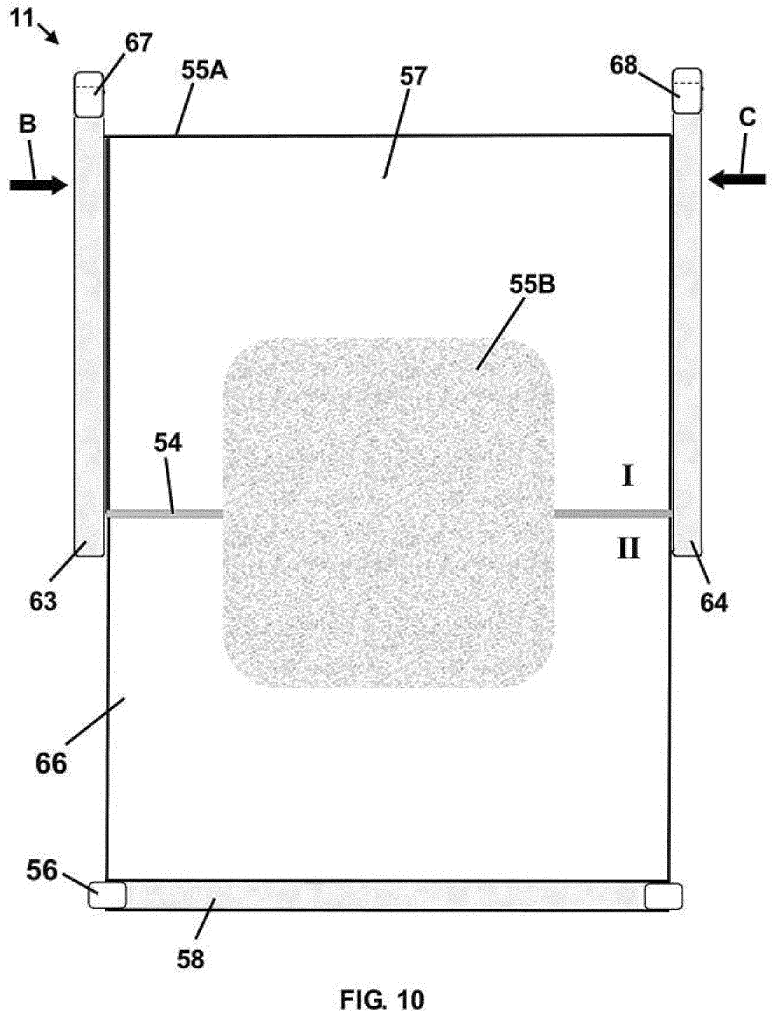

As understood by the skilled artisan, the closed side 53 suitably defines a midline 54 of the changing pad 11 when the changing pad 11 is set to a fully open position as shown in FIG. 10. In this embodiment, the midline 54 further defines first and second Sections I and II of the changing pad 11.

The changing pad 11 of this embodiment suitably includes a first fluid impermeable layer 55A and a second fluid adsorbing layer 55B for absorbing liquid feces and urine, the second fluid adsorbing layer 55B being disposed along the inner surface 57 of the changing pad 11. As shown, in one embodiment a midline of the second fluid adsorbing layer 55B may be linearly aligned along midline 54 or it may be aligned differently as desired. The changing pad 11 may also include one or more additional layers of materials disposed along its inner surface 57 as desired. During use, the inner surface 57 of the changing pad 11 may also be referred to as an upper surface of the changing pad 11 whereby the lower surface is operationally configured to directly abut a support surface, e.g., a countertop, table top, a floor.

In regard to the changing pad 11 as shown in FIG. 10, one suitable changing pad 11 may include a first fluid impermeable layer 55A having a length of 60.96 cm (24.0 inches), a width of 40.64 cm (16.0 inches) and a thickness of 0.5 mm (0.02 inches). Other sizes are herein contemplated as the changing pad 11 is scalable.

With further reference to FIGS. 9 and 10, the accessible sides 50-52 include borders with adhesive surfaces disposed lengthwise along each side of the inner surface 57, i.e., inner mating surfaces. In particular, opposing sides 50 and 51 include mating surfaces with peelable members 60 and 61 or “pull strips” operationally configured to be removed from the changing pad 11 exposing opposing adhesive mating surfaces 63 and 64. In one embodiment, the changing pad 11 may be provided in a folded state with the peelable members 60, 61 being located adjacent the main body 66 of the changing pad 11. Removal of the peelable members 60, 61 exposes the adhesive mating surfaces 63 and 64. In another embodiment, the peelable members 60, 61 may be attached to the main body 66 of the changing pad 11 via perforated type seams in a manner effective to maintain the changing pad 11 in a folded position. In addition, the accessible side 52 may include a peelable member 56 operationally configured to be removed from the changing pad 11 exposing and adhesive mating surface 58. As understood by the skilled artisan, the peelable members 56, 60 and 61 are suitably not removed to expose their corresponding adhesive mating surfaces 58, 63 and 64 until diaper changing activities are completed. Removable of the peelable members 56, 60 and 61 prior to changing of a diaper may result in one or more of the adhesive mating surfaces 58, 63 and 64 adhering to the target individual whose diaper is being changed when the individual is set atop the changing pad 11.

Following use of the changing pad 11, the outer packaging 12, the one or more child changing items and a soiled diaper may be set atop the inner surface 57 along Section I of the outer packaging 12 where after Section II may be folded over to a closed position similar as shown in FIG. 9 whereby the adhesive border 58 suitably adheres to the corresponding opposing inner surface to form side 52. Once sealed, the adhesive surfaces 63 and 64 may be folded inward (see directional arrows B and C) to form seals along sides 50 and 51 thereby sealing the outer packaging 12, one or more child changing items and soiled diaper therein for disposal of the changing pad 11 and its contents. As further shown, the adhesive surfaces 63 and 64 may include distal ends 67 and 68 operationally configured to fold and adhere to the outer surface (or lower surface) of the changing pad 11 after being folded inward as described above. The distal ends 67 and 68 may also include separate peelable members.

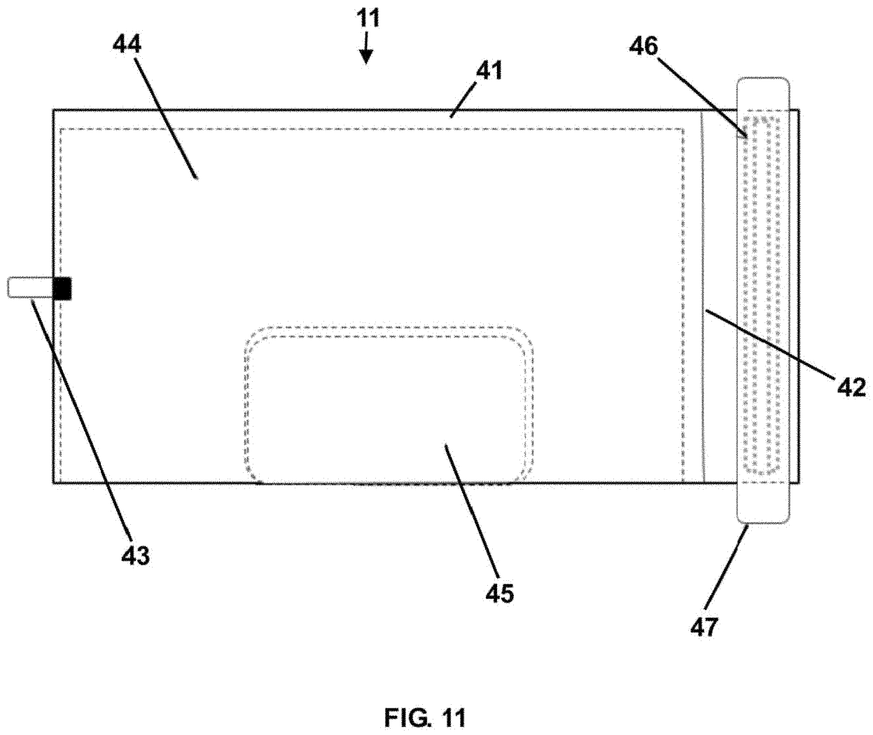

Turning to the simplified illustration of FIGS. 11-14, another embodiment of a changing pad 11 operationally configured as both (1) a diaper changing pad and as (2) a post-change sealable bag for hygienic disposal of the outer packaging 12, the one or more child changing items and a soiled diaper housed therein is provided. As shown, the changing pad 11 includes a first rectangular bag section 41 providing a first usable diaper changing surface. The bag section 41 is suitably defined by a pocket type configuration, e.g., similar to an envelope configuration, with an open first end 42 providing entry within the changing pad 11 and a pull tab inverter 43, or pull string member, located at an opposing sealed second end of the changing pad 11 disposed within the changing pad 11 for grasping purposes. Suitably, the pocket of the bag section 41 has a volume larger enough to accommodate the hand of an adult individual.

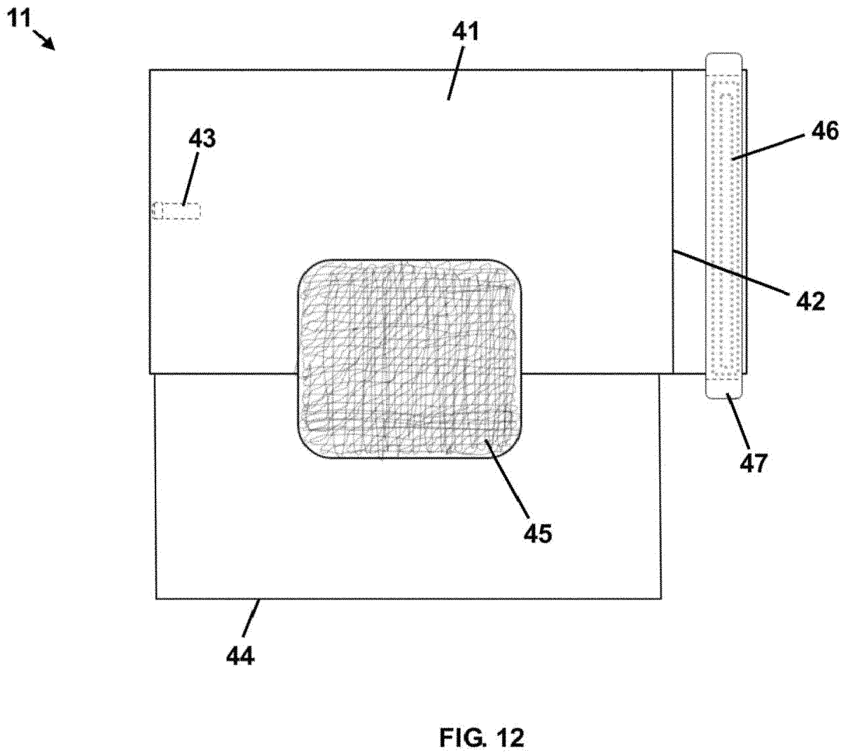

The changing pad 11 may also include a second extendable section or apron 44, which is shown in a folded pre-use position in FIG. 11. The changing pad 11 may also include (1) one or more absorbent members 45 for absorbing liquid feces and urine and (2) one or more adhesive strips 46 with removable covers 47 for sealing the changing pad 11 for disposal purposes. As shown in FIG. 12, when the apron 44 is opened the changing pad 11 provides a substantially planar changing pad for placing atop a support surface when cleaning a child and/or when changing his/her diaper. Without limiting the changing pad 11 to a particular embodiment, a suitable apron 44 is effective to increase the usable surface area of the changing pad 11 as a changing pad up to about 100.0 percent compared to the changing item 40 with the apron 44 is in a folded position as shown in FIG. 11. In addition, the one or more absorbent members 45 may be disposed across parts of both the bag section 41 and the apron 44 as desired for maximizing absorbance coverage.

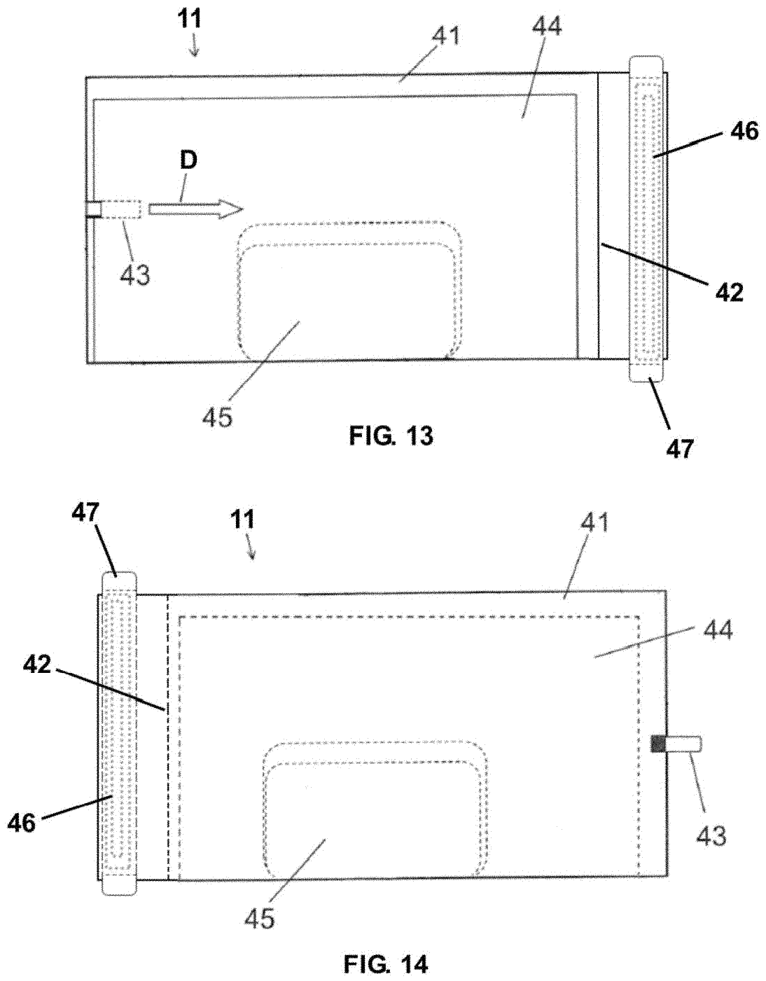

Turning to FIG. 13, once a child's diaper is changed and the child's skin has been wiped down and treated with one or more child changing items, the soiled or used diaper and/or other undergarment (not shown) may be placed atop the open changing pad 11 in a manner effective whereby the apron 44 may be folded to cover such items underneath and simply dispose of the changing pad 11 and its contents. In a more hygienic approach, once the apron 44 is folded with items there under, an individual may stick his/her hand inside the open first end 42 and pull the pull tab inverter 43 toward the open first 42 (see directional arrow D) until the changing pad 11 is turned inside out whereby the apron 44, the soiled diaper, the accessory packaging and used child changing items are enclosed within the changing pad 11 as depicted in FIG. 14. Thereafter, the cover 47 may be removed exposing the adhesive strip 46, which may be folded onto the outer surface of the bag section 41 thereby sealing the open first end 42 of bag section 41 for disposal of the changing pad 11.

As previously stated, a diaper 14 of the present system 10 is disposable and may be operationally configured to adjustably fit children of different torso sizes and shapes from ages ranging from about newborn to about three years of age. In addition, a suitable diaper 14 is operationally configured to shrink or be reduced to a desired size under vacuum. Although the degree of size reduction may vary depending on materials of construction of the diaper 14 and the vacuum applied to its packaging, as stated above one suitable adjustable diaper 14 may be reduced in size under vacuum up to or about 55.0 percent compared to its normal resting size. For example, an adjustable diaper 14 having the following dimensions at a resting size:
Length: about 17.78 cm (about 7.0 inches)
Width: about 13.97 cm (about 5.5 inches)
Height: about 2.54 cm (about 1.0 inches)
   may be reduced to a second size under vacuum (vacuum size) to the following dimensions:
Length: about 15.24 cm (about 6.0 inches)
Width: about 11.43 cm (about 4.5 inches)
Height: about 0.625 cm (about 0.25 inches).

As understood by persons of ordinary skill in the art, a suitable diaper 14 may include a core region for absorbing and storing liquid feces and urine and a chassis for supporting the core region. One suitable core region may include (1) a topsheet that comes in contact with a person's skin, (2) a distribution layer for moving liquid away from a person's skin, and (3) an absorbent core or inner-most section for trapping liquids and holding liquids apart from a person's skin. Although an adjustable diaper 14 for use herein is not necessarily limited to any one combination of materials, one or more suitable materials of diaper 14 construction are presented in Table 1 below.

TABLE 1

| | |
|---|---|
| 1) | Polyethylene film |
| 2) | Polypropylene Non-woven Hydrophobic and Hydrophilic |
| 3) | Absorbent core - Wood pulp fluff |
| 4) | Super Absorbent Polymer |
| 5) | Elastics Lycra/Spandex |
| 6) | Quick wicking layer |
| 7) | Adhesive Hot melts and Elastomeric |
| 8) | Fastening tape - stick type and/or hook and loop type |
| 9) | Moisturizer lotions and fragrance lotions |
| 10) | Breathable/Cloth-like back sheet |

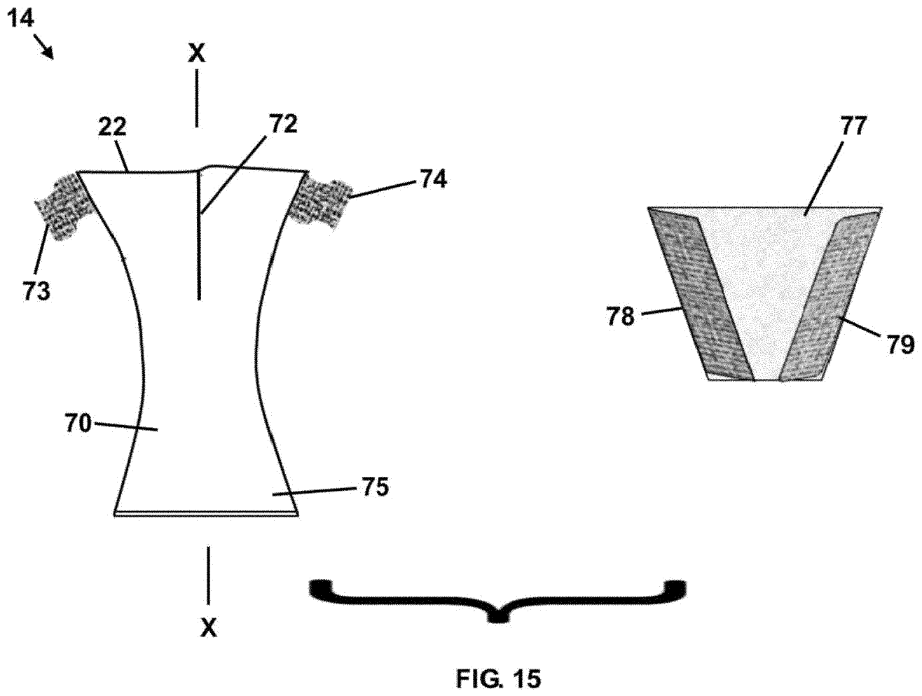

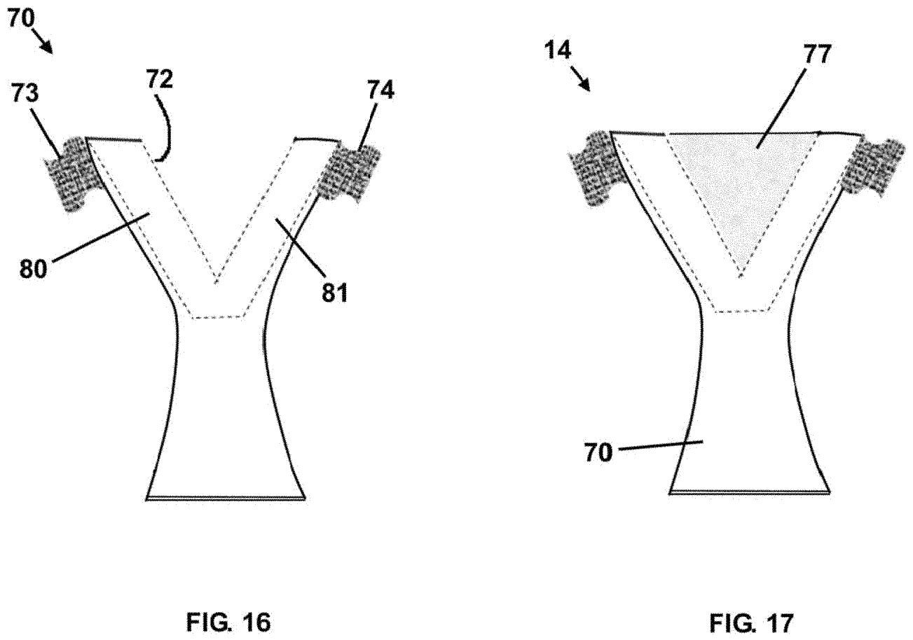

Turning to FIGS. 15-21, one suitable disposable adjustable diaper 14 of the present system 10 may include a main body 70 defined by a split region or slit 72 operationally configured to adjust the width of the diaper 14 from a minimum width (i.e., a closed split region) as shown in FIG. 15 to a maximum width of a fully spread or fully open slit 72 as shown in FIG. 16. As understood by the skilled artisan, the main body 70 may be set at a plurality widths between the minimum and maximum width. The ability to change the width allows for adjustment of the diaper 14 to fit various size individuals. A suitable slit 72 may be located at a midline X-X of the diaper 14 and extend from a first edge 22 of the diaper 14 up to about half the length of the diaper 14. In the embodiment of FIG. 15, the slit 72 extends out about two-fifths the length of the diaper 14.

As shown in FIG. 15, the main body 70 also includes elastic hook strips 73, 74 (or “fastener tabs”) securable to the opposing end 75 of the diaper 14 when the diaper 14 is being worn by an individual. The diaper 14 also includes a co-planar spacer or removable member 77 operationally configured to cover the slit 72 and to operate as the adsorption material of the diaper 14 and form a seamless or continuous diaper 14 as shown in FIG. 17. As shown, the removable member 77 suitably includes opposing sides 78, 79 defined by soft hook material for securing the removable member 77 to corresponding areas 80, 81 adjacent the slit 72 in a continuous manner for diaper 14 usage.

The main body 70 of this embodiment may be constructed from one or materials as described above. In addition, the elastic hook strips 73, 74 may comprise hooks, loops, an adhesive, a cohesive, or other fastening member. For example, the elastic hook strips 73, 74 may be constructed from tape as described in U.S. Pat. No. 5,176,671, titled “Fastening System for Disposable Diaper with Disposability Feature,” issued on Jan. 5, 1993, which is incorporated herein by reference in its entirety. The elastic hook strips 73, 74 may also be constructed from hook and loop type fastening materials as described in U.S. Patent Publication No. 2008/0097368A1, titled “Release Tape-free Fasteners and Disposable Absorbent Articles Utilizing the Same,” published on Apr. 24, 2008; and U.S. Pat. No. 8,221,379 titled “Absorbent Article with Improved Tear Resistance and Softness,” issued on Jul. 17, 2012, each of which is herein incorporated by reference in its entirety.

In one embodiment, the removable member 77 may be constructed from like material(s) as the corresponding main body 70. In another embodiment, the removable member 77 may be constructed from completely different material(s) than the main body 70. In still another embodiment, the removable member 77 may be constructed from one or more like material(s) as the main body 70. Suitably, areas 80 and 81 are constructed from one or more materials effective to receive the soft hook material of opposing sides 78, 79 in a secured manner for desired diaper 14 operation.

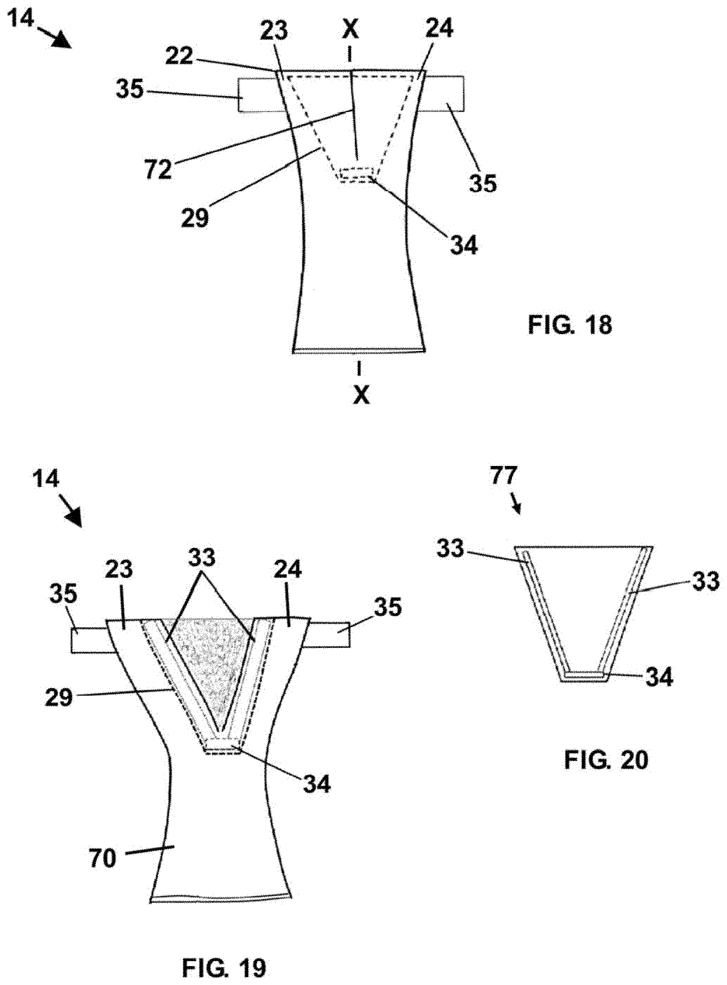

Turning to FIGS. 18-20, in another embodiment a disposable adjustable diaper 14 may include fastening tape 35, as is common to diapers, and a slit 72 similar as described above allowing for adjustment of the diaper 14 to fit a particular size person. Operatively, the slit 28 divides the first edge 22 of the diaper 14 into a first wing 23 and a second wing 24 whereby the width of the first edge 22 of the diaper 14 may be adjusted by adjusting the location of the wings 23, 24 as desired.

When stored in the outer packaging 12, the diaper 14 is suitably set in an unadjusted position (or starting position) with the wings 23, 24 in abutment as depicted in FIG. 18. Once the diaper 14 is removed from the outer packaging 12 the wings 23, 24 may be manipulated as illustrated in FIG. 19 setting the diaper 14 in what may be referred to as an adjusted position. Suitably, the diaper 14 may be adjusted from a minimum size unadjusted position defined by a closed slit 72 (see FIG. 18) to one or more positions defined by an open slit 72 including a maximum adjusted position as described above.

A diaper 14 of this embodiment may be constructed from one or more materials effective for the wings 23, 24 to be easily pulled apart from one another forming a space there between—see FIG. 19 where the separated wings 23, 24 form a V-shape gap of the diaper 14. Similar as the removable member 77 described above, to compensate for this space between the wings 23, 24, the removable member 77 of this embodiment is operationally configured to (1) hold the wings 23, 24 in a fixed position, (2) bridge the gap formed between the separated wings 23, 24 providing a continuous or seamless diaper 14, (3) provide a fluid seal between the wings 23, 24 and the removable member 77, (4) provide comfort to a person's skin during use and (5) operate in similar fashion as the remaining diaper 14 material with regard to absorbing and storing fluid.

Although the removable member 77 may be provided in a size and shape as desired, one suitable removable member 77 has a shape corresponding to the shape formed by the wings 23, 24 when the wings 23, 24 are separated at an open position (see FIG. 19). As such, the maximum size of the adjustable diaper 14 may be determined according to the size and/or shape of the removable member 77.

In one embodiment, the removable member 77 may be releasably attached to the outer surface of the diaper 14 in a manner effective to provide a seal there between. In another embodiment, a distal end of the removable member 77 may be fixed to the diaper 14 near the distal end of the slit 72 (see the removable member 77 attachment point 34) providing a flap like removable member 77 operationally configured to be directed away from the diaper 14. The proximal end of the removable member 77 may be configured as desired. One suitable removable member 77 may include a proximal end similar to the corresponding first edge 22 of the diaper 14. For example, the proximal end of the removable member 77 may include an elastic band corresponding to an elastic band along the first edge 22 of the diaper 14. In one suitable embodiment, part of the main body 70 of the diaper 14 may be constructed from fine loop fabric effective for receiving at least part of the removable member 77 in attachment thereto—the removable member 77 including one or more materials of construction operable with the fine loop fabric.

As understood by the skilled artisan, the removable member 77 may be constructed from one or more like materials as a corresponding main body 70, e.g., having an inner layer, an absorbent core and a waterproof outer layer. As such, when the removable member 77 is disposed between the wings 23, 24 as shown in FIG. 19 the removable member 77 is operationally configured to absorb fluid and provide a soft contact material against a person's skin in a like manner as the corresponding main body 70 of the diaper 14 material. Although the removable member 77 is shown and discussed in terms of being attachable to the outer surface of a main body 70, it is contemplated that the removable member 77 may also be located along the inner surface of a main body 70.

As shown, the removable member 77 is attached along the outer surface of the main body 70. In an embodiment where the diaper 14 has a plastic outer surface, the removable member 77 may include one or more adhesive surfaces for releasable attachment to the outer surface of the wings 23, 24 of the main body 70. As depicted in FIGS. 18-20 including a diaper 14 having a fabric outer layer with a raised pile type surface, the removable member 77 may include one or more attachment members 33, e.g., hook type strips 33, for catching the raised loops of the outer surface of the main body 70 and attaching the removable member 77 to the wings 23, 24 in a manner effective to hold the wings 23, 24 in a fixed position for setting the diaper 14 to a particular size to be worn by a particular size person. Without limiting the hook strips 33 to a particular type, suitable hook strips 33 for use with the diaper 14 of the present application may include soft and ultra-soft hook material as understood by the skilled artisan.

As shown, the one or more attachment members 33 are suitably disposed along the sides of the removable member 77 for securing the sides of the removable member 77 to the outer surface of the wings 23, 24. In operation, a caregiver can adjust the diaper 14 by pulling the removable member 77 apart from the wings 23, 24 so that the wings 23, 24 may be separated to establish a desired width of the first edge 22 of the diaper 14. Once a desired width is established, the removable member 77 can be placed back against the wings 23, 24 fixing the wings in a set position providing a diaper 14 having a size for a particular size person. As such, the adjustable diaper 14 as presented in FIGS. 18-20 is operationally configured to be adjusted in size according to planar spacing of the wings 23, 24, i.e., “planar adjustment,” as opposed to forming folds in the diaper material or forming overlapping sections of diaper material. In other words, the diaper 14 of FIGS. 18-20 may be adjusted without having to increase the thickness of the diaper 14 compared to the same diaper 14 in an unadjusted state.

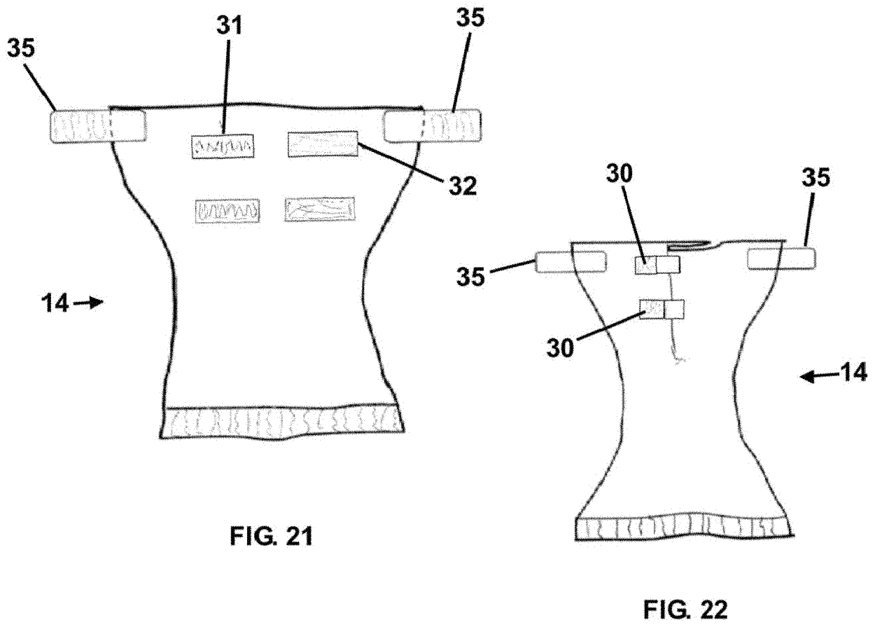

In an alternative embodiment as shown in FIGS. 21 and 22, a disposable adjustable diaper 14 may include one or more adjustment members 30 and fastening tape 35 effective to hold the diaper 14 in an operable position until the diaper 14 is to be removed from the a wearer. The adjustment members 30 are effective for adjusting the diaper 14 to a particular size person by folding the diaper 14 and mating the adjustment members 30 at particular point along the adjustment members 30 to maintain a diaper 14 in a desired folded position (see FIG. 22). As understood by the skilled artisan, the diaper 14 may be used in a completely unfolded state as shown in FIG. 21 or shaped to a maximum fold according to the configuration of the adjustment members 30 employed. In one embodiment, adjustment members 30 may be provided at a desired length, shape, and width with adhesive surfaces for holding the diaper 14 in a secured folded position when mating corresponding adjustment members 30 together. In another embodiment, adjustment members 30 may be provided as hook 31 and loop 32 fasteners at a desired length, shape, and width for holding the diaper 14 in a secured folded position. Although two pairs of corresponding adjustment members 30 are shown in FIGS. 21 and 22, adjustment members 30 may be provided in one or more pairs as desired or otherwise required. Also, the adjustment members 30 may be arranged along the outside of the diaper 14 or along the inside of the diaper 14 free from tampering. As such, the fold in the diaper 14 may be located on the inside of the diaper 14 or the outside of the diaper 14 as desired.

The invention will be better understood with reference to the following non-limiting example, which is illustrative only and not intended to limit the present invention to a particular embodiment.

### EXAMPLE 1

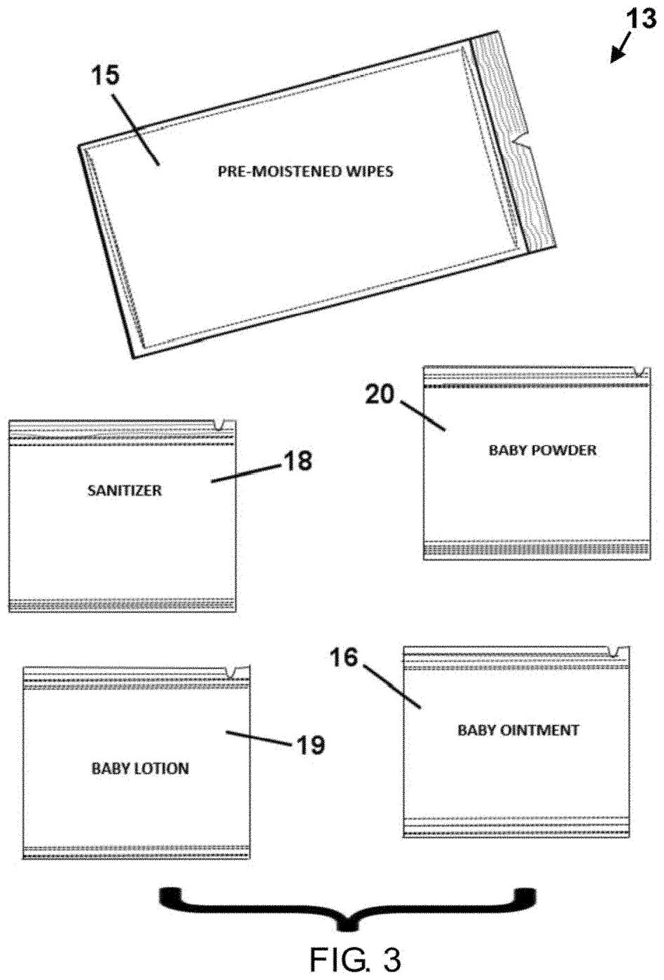

In a first non-limiting example, a system 10 as shown in FIG. 1 including the changing item accessories of FIG. 3, a diaper 14 as described in Table 1 and a changing pad 11 is provided. The system 10 includes a plastic outer packaging 12 having the following dimensions:
Length: about 17.78 cm (about 7.0 inches)
Width: about 12.7 cm (about 5.0 inches)
Height: about 2.54 cm (about 1.0 inches)
   and an adjustable diaper 14 having the following dimensions at a resting size:
Length: about 17.78 cm (about 7.0 inches)
Width: about 13.97 cm (about 5.5 inches)
Height: about 2.54 cm (about 1.0 inches).

Persons of ordinary skill in the art will recognize that many modifications may be made to the present application without departing from the spirit and scope of the invention. The embodiment(s) described herein are meant to be illustrative only and should not be taken as limiting the invention, which is defined in the claims.

We claim:

1. A system for changing out a used absorbent article worn by a person including:
an outer packaging; and
a clean adjustable absorbent article housed within the outer packaging operationally configured to fit various size persons.

2. The system of claim 1 further including a disposal changing pad housed within the outer packaging operationally configured as a changing surface and as a stand-alone disposal bag for disposal of the outer packaging, the one or more child changing items and the used absorbent article.

3. The system of claim 1 wherein the outer packaging is vacuum shrunk and the clean adjustable absorbent article includes a first normal resting size and a second reduced size under vacuum up to or about 55.0 percent that of the first normal resting size of the absorbent article.

4. The system of claim 1 wherein the clean adjustable absorbent article includes a diaper including a split region operationally configured to adjust the width of the diaper from a minimum width of a closed split region to a maximum width of a fully open split region.

5. A diaper changing system including:
an outer packaging;
one or more diaper changing accessory items housed therein including at least a diaper changing pad and a vacuum shrunk adjustable diaper, wherein the diaper changing pad is operationally configured as a post diaper change disposal bag for disposal of at least the outer packaging and a soiled diaper.

6. The system of claim 5 wherein the diaper changing accessory items further include one or more pre-moistened wipes and one or more skin care items.

7. The system of claim 5 wherein the diaper changing pad is a foldable planar type member with at least a first fluid impermeable layer and a second fluid adsorbing layer.

8. The system of claim 5 wherein the adjustable diaper includes (1) a main body including a split region operationally configured to adjust the width of the diaper from a minimum width of a closed split region to a maximum width of a fully open split region and (2) a removable member operationally configured to cover the split region.

9. A method of changing a child's diaper including:
providing a diaper changing system including an outer packaging and items housed therein including at least an adjustable diaper, a diaper changing pad and one or more wiping type items;
removing the items from the outer packaging and placing the diaper changing pad on a support surface in an open position;
placing a child wearing a used diaper atop the diaper changing pad;
removing the used diaper and cleaning the child using the one or more wiping type items;
placing the adjustable diaper on the child in a fitted manner; and
sealing the outer packaging, the used diaper and one or more wiping type items within the diaper changing pad for disposal.
